(19) United States
(12) Patent Application Publication
(10) Pub. No.: US 2010/0298603 A1
(43) Pub. Date: Nov. 25, 2010

(54) PROCESS FOR PRODUCING 2-HYDROXY-4-METHYLTHIOBUTANEAMIDE

(76) Inventors: Takanori Ito, Ehime (JP); Masahiro Kinoshita, Ehime (JP)

Correspondence Address: BIRCH STEWART KOLASCH & BIRCH, PO BOX 747, FALLS CHURCH, VA, 22040-0747, US

(21) Appl. No.: 12/812,548

(22) PCT Filed: Jan. 9, 2009
(86) PCT No.: PCT/JP2009/050600

§ 371 Date: Jul. 12, 2010

(30) Foreign Application Priority Data
Jan. 10, 2008 (JP) 2008-002989

(51) Int. Cl. C07C 319/20; C07C 231/06

(52) U.S. Cl. 562/581; 564/129

(57) ABSTRACT

Provided is a process for producing an amide (A): by hydrating a nitrile (B) at high conversion in relatively short period of time even without use of high-capacity cooling apparatuses (25, 35) and a large amount of inorganic acid (D). In the process for the present invention, a nitrile (B) is hydrated in continuous mode in the presence of an inorganic acid (D) so as to give a conversion of 80% to 98%, and the unreacted nitrile contained in the resultant hydrated reaction liquid (E) is hydrated in batch-wise mode so as to give a conversion of 99.9% or more, thereby producing an amide (A). For example, the inorganic acid (D) is sulfuric acid and the use amount thereof is 0.5 to 1-fold mol with respect to the nitrile (B), and the temperature in hydration is 40 to 70-C, and hydration is performed in continuous mode using a tubular reactor (2c), loop reactor (2d) and the like. The resultant amide (A) can be hydrolyzed to produce a thiobutanoic acid (G).

### TECHNICAL FIELD

The present patent application claims priority under the Paris Convention based on Japanese Patent Application No. 2008-002989 (filed on Jan. 10, 2008), the entire content of which is herein incorporated by reference.

The present invention relates to a process for producing 2-hydroxy-4-methylthiobutaneamide.

### BACKGROUND ART

2-hydroxy-4-methylthiobutaneamide [hereinafter, abbreviated as amide in some cases] is a compound of the formula (A):

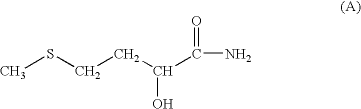

and useful as a production intermediate for 2-hydroxy-4-methylthiobutanoic acid [hereinafter, abbreviated as HMBA in some cases] of the formula (G):

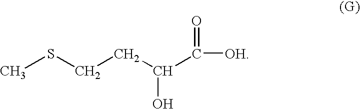

Known as a process for producing this amide (A) is a process for hydrating 2-hydroxy-4-methylthiobutanenitrile [hereinafter, abbreviated as nitrile in some cases]of the formula (B)

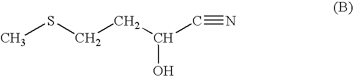

with water [H₂O](C) in the presence of an inorganic acid (D), and patent document 1 [Japanese Patent Application Laid-Open (JP-A) No. 2001-187779, paragraph nos. 0013, 0038, 0046 and 0052] discloses a process for producing an amide (A) in batch-wise mode in which a tank reactor is used as a reaction apparatus, and a nitrile (B), water (C) and inorganic acid (D) are introduced into this reactor and hydrated over a sufficient period of time also after completion of the introduction. In such a process, raw materials such as nitrile and the like are introduced before hydration over a sufficient period of time, thus, 99.9% or more of the nitrile (B) introduced can be hydrated.

If the temperature in hydrating nitrile is high, hydrolysis of the produced amide occurs to generate HMBA, and simultaneously, also ammonia [NH₃] is by-produced. When ammonia is by-produced, it forms a salt with an inorganic acid to consume the inorganic acid, accordingly, hydration does not progress easily and unreacted nitrile tends to remain. Because of this reason, it is preferable to perform hydration at temperatures of 70° C. or lower to prevent hydrolysis of the produced amide, for hydrating the nitrile at high conversion with a small amount of inorganic acid.

However, the nitrile hydration reaction manifests significantly large heat generation value, additionally, the reaction progresses extremely quickly in the early phase of the reaction, and particularly in introduction of nitrile, water and inorganic acid, the temperature tends to drastically increase. Owing to this, it is necessary, for performing hydration while maintaining temperatures of 70° C. or lower, to carry out hydration while extracting heat using a high-capacity cooling apparatus corresponding to drastic temperature rise or to introduce raw materials such as nitrile and the like into a reactor portion-wise over an extremely long period of time.

(Patent document 1) JP-A No. 2001-187779, paragraph nos. 0038, 0046 and 0052

(Non-patent document 1) “Revised 6th Edition, Chemical Engineers' Handbook” (Feb. 25, 1999, published by Maruzen Co. Ltd.), p. 186 to 187

(Non-patent document 2) “Revised 6th Edition, Chemical Engineers' Handbook” (Feb. 25, 1999, published by Maruzen Co. Ltd.), p. 1028 to 1030

As a process capable of carrying out a reaction while suppressing drastic temperature increase due to reaction heat even with a low-capacity cooling apparatus, general is a process for reacting in continuous mode using a continuous reactor, however, for obtaining an amide by hydration of a nitrile at high conversion only by the continuous mode reaction, the nitrile has to be hydrated quickly, leading to necessity of use of a large amount of inorganic acid.

### DISCLOSURE OF INVENTION

The present inventors have intensively investigated to develop a process capable of hydrating a nitrile at high conversion to produce an amide even without use of a high-capacity cooling apparatus and a large amount of inorganic acid, and resultantly found that if a nitrile is hydrated in continuous mode so as to give a conversion of 80% or more and 98% or less and the unreacted nitrile in the resultant hydrated reaction liquid is hydrated in batch-wise mode, then, the use amount of an inorganic acid, in the continuous mode hydration, can be decreased since a nitrile may be hydrated at a relatively lower conversion of 98% or less, and in the batch-wise mode hydration, a drastic progress of the reaction is not observed, heat generation value is small and sufficient heat removal can be carried out even with a low-capacity cooling apparatus, since hydration has been already performed at a conversion of 80% or more, leading to completion of the present invention.

That is, the present invention provides a process for producing 2-hydroxy-4-methylthiobutaneamide (A) by hydration of 2-hydroxy-4-methylthiobutanenitrile (B) in the presence of an inorganic acid (D), comprising hydrating in continuous mode 2-hydroxy-4-methylthiobutanenitrile (B) in the presence of an inorganic acid (D) so as to give a conversion of 80% or more and 98% or less to obtain hydrated reaction liquid (E) and hydrating in batch-wise mode unreacted 2-hydroxy-4-methylthiobutanenitrile contained in the resultant hydrated reaction liquid (E) so as to give a conversion of 99.9% or more. FIGS. 1 to 6 show schematically one example of a production equipment (1) for producing an amide (A) by reaction of a nitrile (B) with water (C) in the presence of an inorganic acid (D) according to the production process for the present invention.

According to the production process for the present invention, a nitrile is first hydrated in a continuous reactor, thus, there is no necessity of use of a high-capacity cooling apparatus and a large amount of inorganic acid, and an amide can be produced at high conversion.

### BRIEF DESCRIPTION OF DRAWINGS

FIG. 1 is a view showing schematically one example of an equipment for producing 2-hydroxy-4-methylthiobutaneamide by the production process for the present invention.

FIG. 2 is a view showing schematically one example of an equipment for producing 2-hydroxy-4-methylthiobutaneamide by the production process for the present invention.

FIG. 3 is a view showing schematically one example of an equipment for producing 2-hydroxy-4-methylthiobutaneamide by the production process for the present invention.

FIG. 4 is a view showing schematically one example of an equipment for producing 2-hydroxy-4-methylthiobutaneamide by the production process for the present invention.

FIG. 5 is a view showing schematically one example of an equipment for producing 2-hydroxy-4-methylthiobutaneamide by the production process for the present invention.

FIG. 6 is a view showing schematically one example of an equipment for producing 2-hydroxy-4-methylthiobutaneamide by the production process for the present invention.

FIG. 7 is a view showing schematically one example of a continuous reactor.

FIG. 8 is a view showing schematically one example of a continuous reactor.

FIG. 9 is a view showing schematically one example of a continuous reactor.

### EXPLANATION OF MARKS

A: 2-hydroxy-4-methylthiobutaneamide (amide)
B: 2-hydroxy-4-methylthiobutanenitrile (nitrile)
C: water D: inorganic acid D′: inorganic acid aqueous solution (water+inorganic acid)
E: hydrated reaction liquid F: water
G: 2-hydroxy-4-methylthiobutanoic acid (HMBA)
1: production equipment
2: continuous reactor
2a: continuous stirred tank reactor
20a: tank reactor body
2b: serial continuous stirred tank reactor
2c: tubular reactor
20c: tubular reactor body
2d: loop reactor
20d: loop reactor body
21: nitrile introduction tube
22: inorganic acid aqueous solution introduction tube
23: jacket 24: pump 25: cooling apparatus
26: stirred 27: extraction tube
3,3′: batch-wise reactor
30: tank reactor body
33: jacket 34: pump 35: cooling apparatus
36: stirred 37: extraction tube 38: water introduction tube
4: hydrated reaction liquid storing tank
43: jacket 44: pump 45: cooling apparatus

### BEST MODE FOR CARRYING OUT THE INVENTION

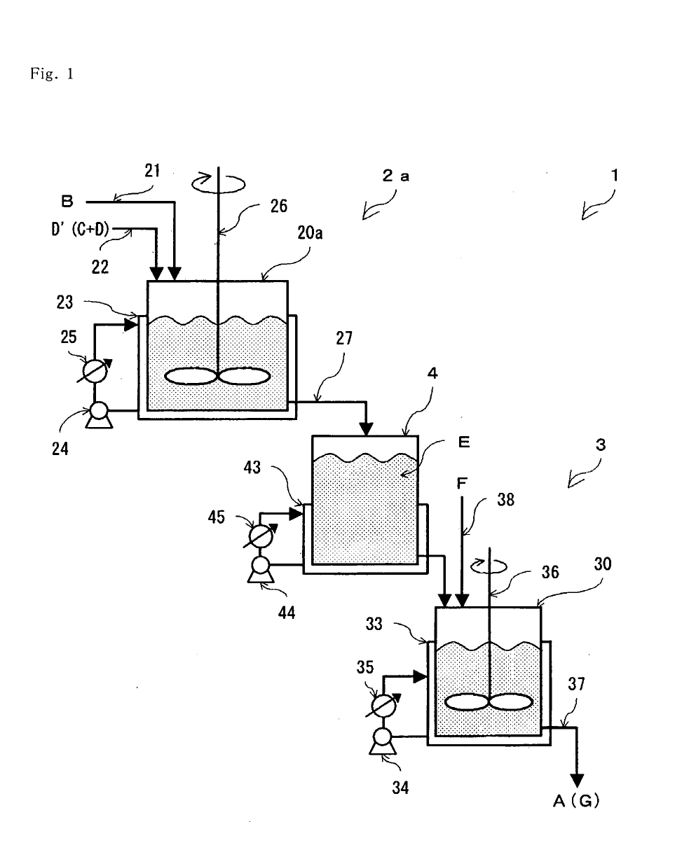

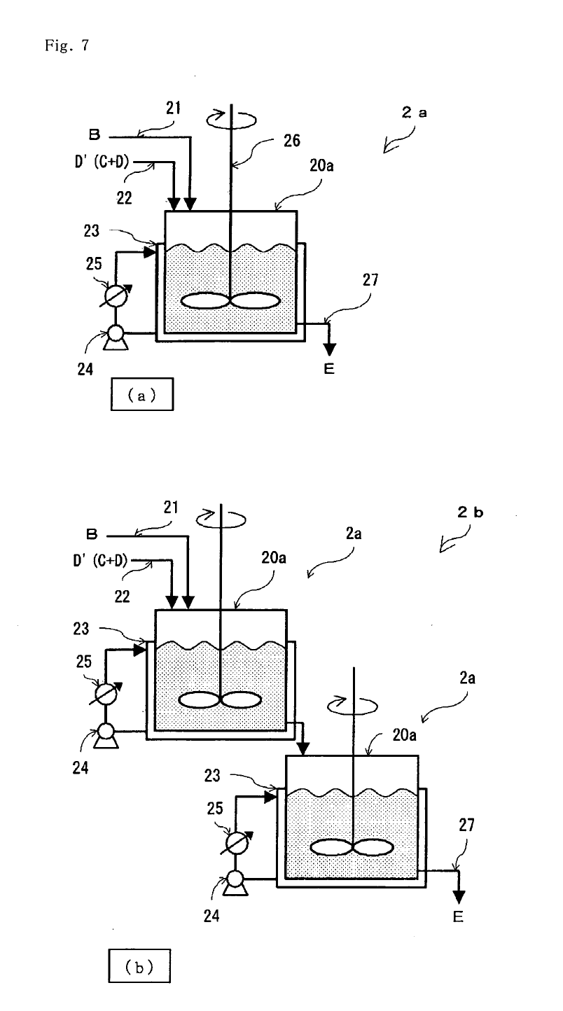

The production process for the present invention will be illustrated below using FIGS. 1 to 9. A production equipment (1) shown in FIGS. 1 to 6 is used for producing an amide (A) according to the production process for the present invention, and equipped with a continuous reactor (2) and a batch-wise reactor (3). FIGS. 7 to 9 show schematically examples of the continuous reactor (2) which can be used in this production equipment (1).

The process for the present invention is a process for producing 2-hydroxy-4-methylthiobutaneamide (A) by hydrating 2-hydroxy-4-methylthiobutanenitrile (B). The use amount of water (C) to be used for hydration may be stoichiometrically 1-fold mol or more with respect to the nitrile (B), and preferably 0.1-fold weight or more, and usually 0.4-fold weight or less.

The nitrile hydration is carried out in the presence of an inorganic acid (D). As the inorganic acid, for example, sulfuric acid is preferably used, and the use amount thereof is usually 0.5-fold mol or more, preferably 0.6-fold mol or more and may over 1-fold mol with respect to the nitrile, and in the production process for the present invention, it is usually 1-fold mol or less, preferably about 0.8-fold mol or less since an amide can be obtained with good conversion even if the amount is 1-fold mol or less.

In the production process for the present invention, first, a nitrile (B) is hydrated in continuous mode in the presence of an inorganic acid (D) to obtain hydrated reaction liquid (E). Usually, the nitrile (B), water (C) and inorganic acid (D) are introduced into a continuous reactor (2), and hydrated in continuous mode in this continuous reactor (2). The continuous reactor (2) is a reactor for reacting the nitrile (B) with water (C) in the presence of the inorganic acid (D) by a continuous operation.

The nitrile (B) is introduced into the continuous reactor (2) without mixing with the inorganic acid, alternatively, the nitrile (B) is previously mixed with a portion of the inorganic acid (D) before introducing into the continuous reactor (2). The nitrile (B) may be previously mixed with water (C) to provide a nitrile aqueous solution which is then introduced, or may be introduced without mixing with water (C), and depending on the production process for the nitrile (B), water contained in the production process of nitrile may be maintained in introduction.

Though the inorganic acid (D) and water (C) may be each independently introduced into the continuous reactor (2), they are usually mixed previously and introduced in the form of an inorganic acid aqueous solution (D′).

The continuous reactor (2) includes, for example, a continuous stirred tank reactor (CSTR) (2a) in which a tank reactor body (20a) is used singly, into this is introduced a nitrile (B), water (C) and inorganic acid (D) continuously, and hydrated reaction liquid (E) in the reactor is continuously extracted while stirring, as shown in FIG. 7 (a) [non-patent document 1: “Revised 6th Edition, Chemical Engineers' Handbook” (Feb. 25, 1999, published by Maruzen Co. Ltd.), p. 186 to 187]. The continuous stirred tank reactor (2a) shown in FIG. 7 (a) has a nitrile introduction tube (21) for introducing a nitrile (B) into the body (20a), and an inorganic acid aqueous solution introduction tube (22) for introducing water (B) and inorganic acid (D) in the form of an inorganic acid aqueous solution (D′). The body (20a) is covered with a jacket (23), and cooled by a cooling medium in this jacked (23). The cooling medium is circulated by a pump (24), and cooled by a cooling apparatus (25). The body (20a) has a stirrer (26), and the nitrile (B), water (C) and inorganic acid (D) introduced react while being stirred by this stirrer (26). The resultant hydrated reaction liquid (E) is continuously extracted from an extraction tube (27). FIG. 1 shows an example using this continuous stirred tank reactor (2a) as the continuous reactor (2).

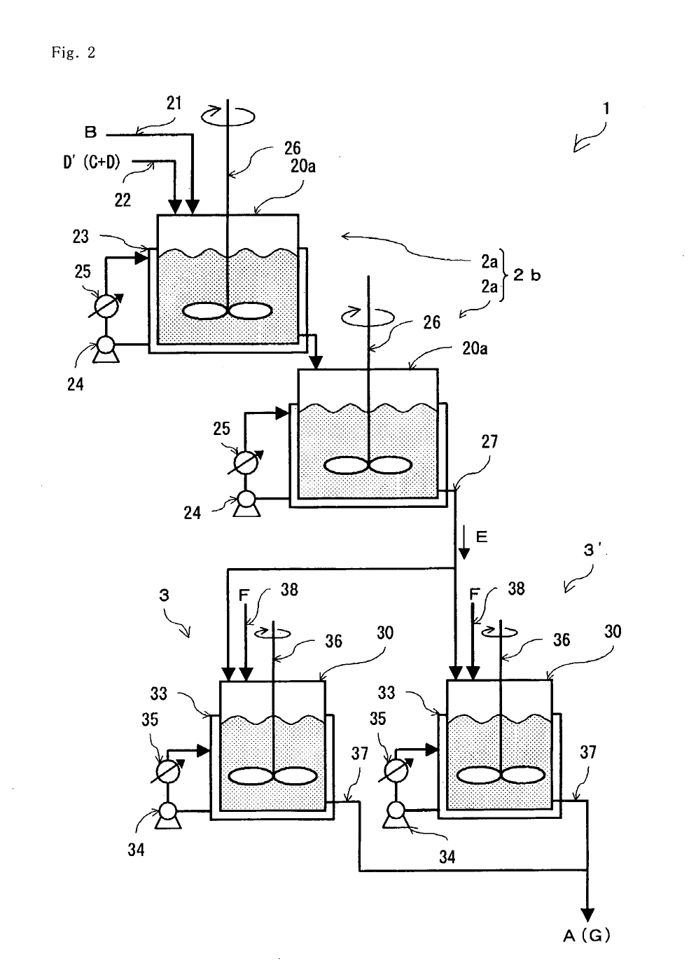

The continuous reactor (2) includes also a serial continuous stirred tank reactor (2b) in which two or more of the above-described continuous stirred tank reactors (2a) are used and these are connected serially, as shown in FIG. 7 (b) [non-patent document 1: “Revised 6th Edition, Chemical Engineers' Handbook” (Feb. 25, 1999, published by Maruzen Co. Ltd.), p. 186 to 187]. In the serial continuous stirred tank reactor (2b), usually about two to five of the continuous stirred tank reactors (2a) are connected serially. FIG. 7 (b) shows an example of two serial connection. FIG. 2 shows an example using this serial continuous stirred tank reactor (2b) as the continuous reactor.

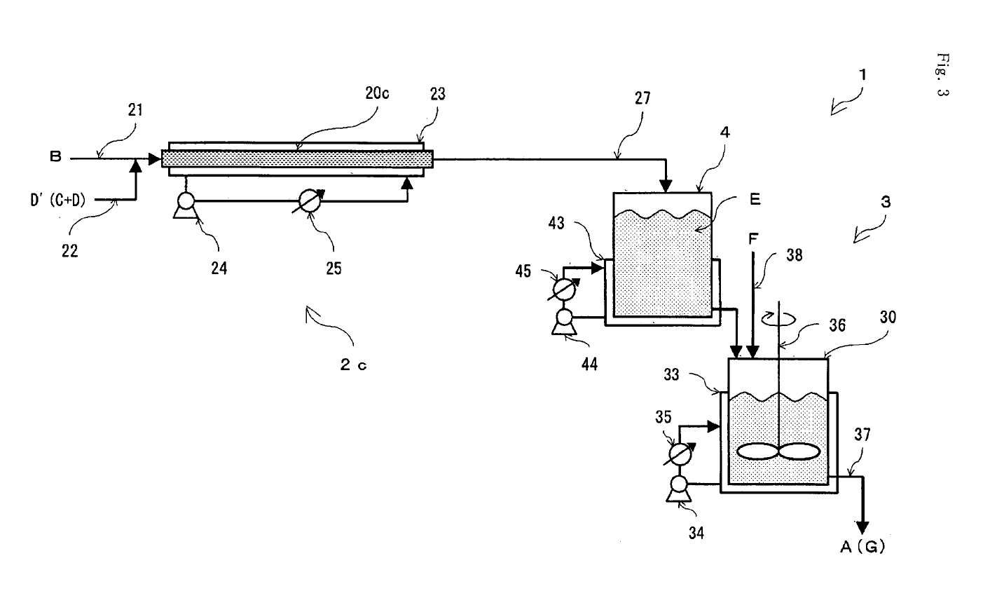

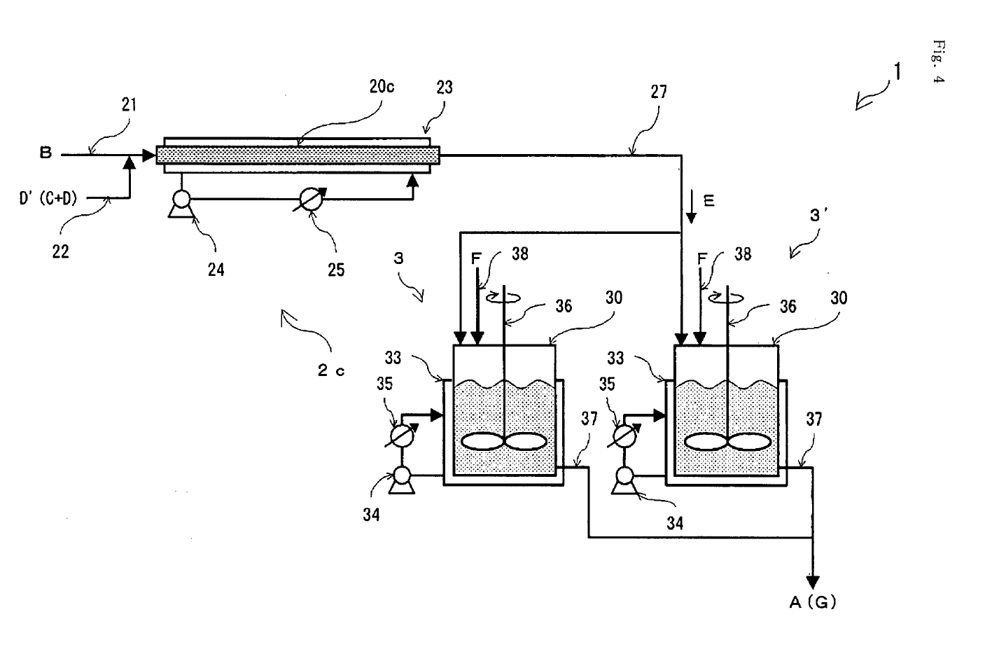

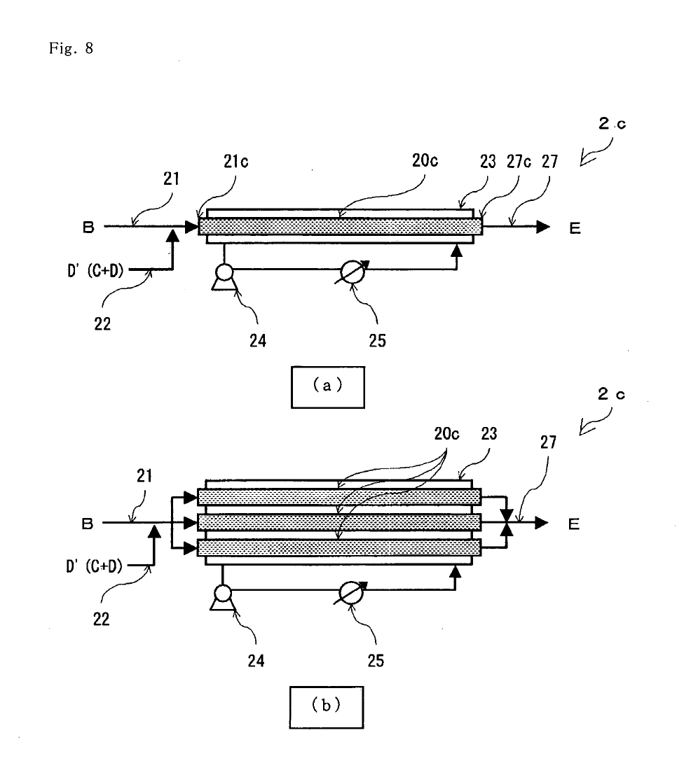

The continuous reactor (2) includes also a tubular reactor (2c) as shown in FIG. 8 (a). The tubular reactor (2c) is a reactor having a tubular reactor body (20c) in which a nitrile (B), water (C) and inorganic acid (D) are continuously introduced from its one end (21c) and reacted while passing through toward another end (27c), and extracted continuously from the another end (27c), and examples thereof include a plug flow reactor and the like. The tubular reactor (2c) shown in FIG. 8 (a) has a reactor body (20c), a nitrile introduction tube (21) and inorganic acid aqueous solution introduction tube (22) connected to on end (21c) of the reactor body, and an extraction tube (27) connected to another end (27c) of the reactor body, and the periphery of this reactor body (20c) is covered with a jacket (23). This jacked (23) is filled with a cooling medium, and for example, this cooling medium is cooled by a cooling apparatus (25) while circulating by a pump (24), thereby removing hydration heat generated by hydration in the reactor body (20). In FIG. 8 (a), a straight tubular body is exemplified as the tubular reactor body (20), however, it may be a coiled body. The tubular reactor (2c) may take a constitution in which a plurality of tubular reactor bodies (20c) are connected in parallel and covered with a jacked (23), as shown in FIG. 8 (b). FIGS. 3 and 4 show examples using this tubular reactor (2c) as the continuous reactor.

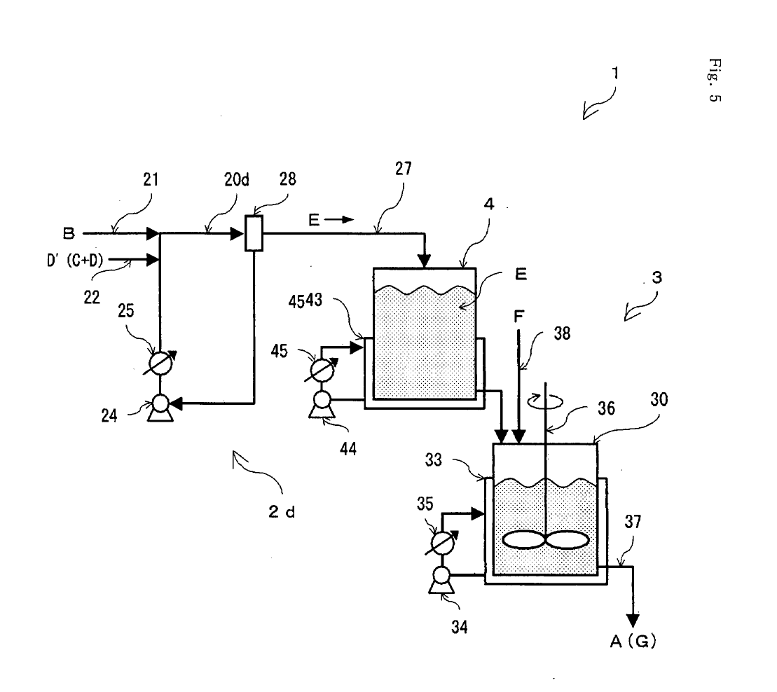

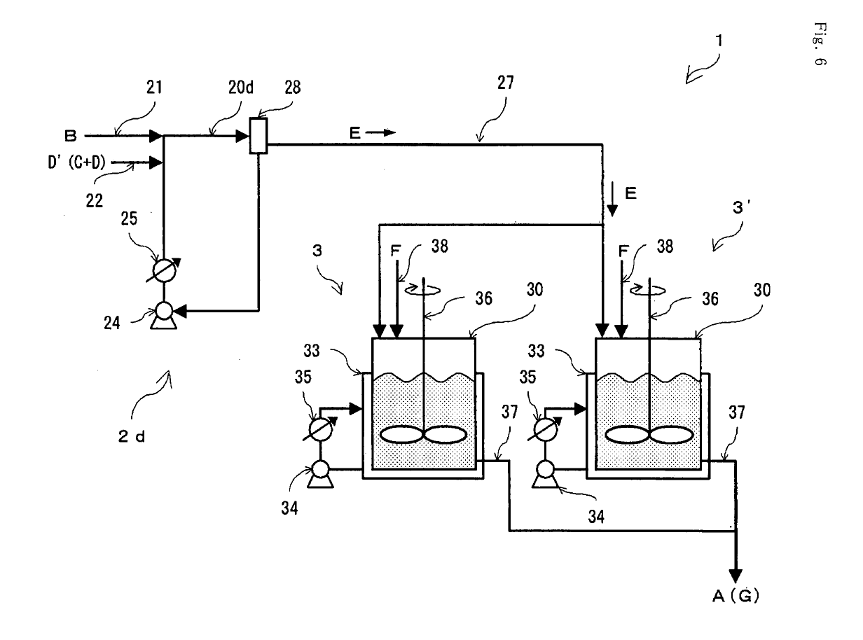

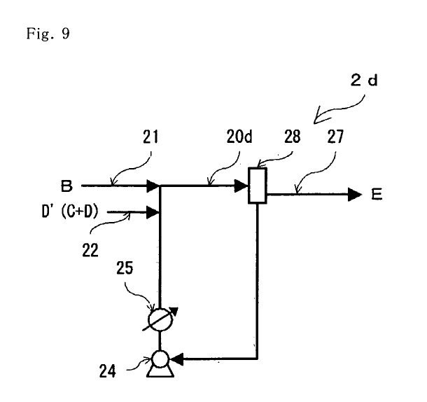

The continuous reactor (2) includes also a loop reactor (2d) as shown in FIG. 9. The loop reactor (2d) is a reactor having a loop tube (20d) in which reaction liquid circulates, wherein a nitrile (B), water (C) and inorganic acid (D) are continuously introduced into this loop tube (20d) and hydrated continuously while circulating in the loop (20d), and the reaction liquid is extracted continuously from the loop tube (20d), thereby reacting them continuously [(non-patent document 2) “Revised 6th Edition, Chemical Engineers' Handbook” (Feb. 25, 1999, published by Maruzen Co. Ltd.), p. 1028 to 1030]. In the loop reactor (2d) shown in FIG. 9, the loop tube (20d) is equipped with a pump (24) and a cooling apparatus (25), and heat is removed by the cooling apparatus (25) while circulating reaction liquid (E) in the loop tube (20) by this pump (24). By the loop reactor (2), the nitrile (B) can be hydrated continuously. To the loop tube (20d), a nitrile introduction tube (21) for introducing the nitrile (B) and an inorganic acid aqueous solution introduction tube (22) for introducing water (B) and inorganic acid (D) in the form of inorganic acid aqueous solution (D′) are connected, and also an extraction tube (27) for extracting a portion of the reaction liquid (E) is connected. Though the extraction tube (27) may be connected directly to the loop tube (20), it is preferable that the extraction tube (27) is connected at a tank part (28) provided in the middle of the loop tube (20) so that extraction in constant liquid amount is possible even if the liquid quantities of the nitrile (B), water (C) and inorganic acid to be introduced into the loop tube (20) vary. FIGS. 5 and 6 show examples using this loop reactor (2d) as the continuous reactor.

By such a continuous reactor (2), the nitrile (B) can be hydrated continuously, and in this continuous reactor (2), hydration is usually performed at hydration temperatures of about 40° C. to 70° C. When the hydration temperature is lower than 40° C., hydration does not progress sufficiently. When over 70° C., there is a tendency that ammonia is by-produced together with HMBA and thus an amide (A) is not obtained easily at high conversion, and this tendency is remarkable when the use amount of an inorganic acid is 1-fold mol or less with respect to the nitrile.

The conversion when hydrating the nitrile (B) continuously is 80% or more, preferably 85% or more and 98% or less, preferably 95% or less. If the conversion of the nitrile is less than 80% and the amount of over 20% of the nitrile introduced remains as the unreacted nitrile without hydration, then, heat generation occurring in the subsequent hydration in batch-wise mode of the unreacted nitrile increases, and in contrast, it is necessary, for hydration of the nitrile in continuous mode at a conversion of over 98%, to increase the use amount of the inorganic acid (D), that is, both of the cases are undesirable.

The conversion of the nitrile (B) in hydrating in continuous mode by the continuous reactor (2) can be controlled by the residence time, in addition to the hydration temperature. When the residence time is shorted, the conversion is lower, and when the residence time is longer, the conversion is higher. For shortening the residence time, it may be advantageous to use the continuous reactor (2) of small content volume, or to increase the introduction quantities of the nitrile (B), water (C) and inorganic acid (D) into the continuous reactor (2). For elongating the residence time, it may be advantageous to use the continuous reactor (2) of large content volume, or to decrease the introduction quantities of the nitrile and the like. The residence time in hydrating the nitrile (B) in continuous mode is usually about 0.2 hours to 2 hours.

The hydrated reaction liquid (E) after hydration of the nitrile (B) is extracted continuously through the extraction tube (27) from the continuous reactor (2). The hydrated reaction liquid (E) extracted contains intended 2-hydroxy-4-methylthiobutaneamide, and unreacted water, inorganic acid and the like, and additionally, unreacted 2-hydroxy-4-methylthiobutanenitrile. Further, HMBA generated by hydrolysis of an amide is contained in some cases.

The hydrated reaction liquid (E) extracted from the extraction tube (27) contains unreacted nitrile, and such a nitrile is hydrated in batch-wise mode. For hydrating the unreacted nitrile in batch-wise mode, the hydrated reaction liquid (E) is introduced into a batch-wise reactor (3) and hydrated in this batch-wise reactor (3) in batch-wise mode. The batch-wise reactor (3) is a reactor for hydrating the unreacted nitrile contained in the hydrated reaction liquid (E) by a batch-wise operation, and for example, a tank reactor (30) is used [non-patent document 1: “Revised 6th Edition, Chemical Engineers' Handbook” (Feb. 25, 1999, published by Maruzen Co. Ltd.), p. 186 to 187].

For hydrating in batch-wise mode the unreacted nitrile, it may be advantageous, for example, to hydrate the unreacted nitrile contained in the reaction liquid by reacting with water, by a batch-wise operation in which the hydrated reaction liquid (E) is introduced into this batch-wise reactor (3), then, the hydrated reaction liquid (E) is reacted while maintaining at the hydration temperature.

The hydrated reaction liquid (E) is extracted continuously from the continuous reactor (2). In contrast, into the batch-wise reactor (3), it is intermittently introduced by a batch-wise operation. Thus, is may be permissible, for example as shown in FIGS. 1, 3 and 5, that a hydrated reaction liquid storing tank (4) is inserted between the continuous reactor (2) and the batch-wise reactor (3), and the hydrated reaction liquid (E) extracted continuously from the continuous reactor (2) is stored continuously in this hydrated reaction liquid storing tank (4) and a necessary amount of the liquid is extracted from this tank (4) and introduced into the batch-wise reactor (3). Since the unreacted nitrile contained in the hydrated reaction liquid (E) is hydrated to become an amide to generate heat in some cases also during storing in the hydrated reaction liquid storing tank (4), it may be permissible that also the hydrated reaction liquid storing tank (4) is provided with a jacket (43), and a cooling medium cooled by a cooling apparatus (45) is circulated into this jacket (43) by a pump (44), thereby removing heat.

Further, it may be permissible, as shown in FIGS. 2, 4 and 6, that two or more batch-wise reactors (3,3′) are used and these batch-wise reactors (3,3′) are used alternately, and during hydration in one reactor (3) of these batch-wise reactors, the hydrated reaction liquid (E) extracted from the continuous reactor (2) is introduced into another batch-wise reactor (3′).

It may also be permissible that hydration progresses also during introduction of the hydrated reaction liquid (E) into the batch-wise reactor (3) from the continuous reactor (2) and during storing in the hydrated reaction liquid storing tank (4), and a portion of the unreacted nitrile (B) contained in the hydrated reaction liquid (E) is hydrated to become an amide.

In the batch-wise reactors (3) shown in FIGS. 1 to 6, a jacket (33) is provided on the periphery of the tank reactor body (30), and a cooling medium from the cooling apparatus (35) can be passed through this jacket (33) by the pump (34) to remove heat.

By this batch-wise reactor (3), the unreacted nitrile can be hydrated in batch-wise mode, and in this batch-wise reactor (3), hydration is carried out usually at hydration temperatures of about 40° C. to 70° C. For hydration at such temperatures, it is usual that hydration is carried out while removing heat by the jacket (33) so that the temperature in the tank reactor body (30) is within this temperature range. When the hydration temperature is lower than 40° C., hydration does not progress sufficiently. When over 70° C., there is a tendency that ammonia is by-produced together with HMBA and thus the nitrile conversion lowers by contraries, and this tendency is remarkable when the use amount of an inorganic acid is 1-fold mol or less with respect to the nitrile.

Hydration is usually carried out under stirring, and in the batch-wise reactors (3) shown in FIGS. 1 and 2, hydration is carried out while stirring by the stirrer (36).

Since the nitrile to be hydrated in the body (30) of the batch-wise reactor is the unreacted nitrile contained in the hydrated reaction liquid (E) after previous hydration of nitrile at a conversion of 80% or more, the hydration speed is relatively mild, the heat generation is slight, and hydration can be carried out while maintaining hydration temperatures of 70° C. or lower even with a cooling apparatus (35) of relative low-capacity.

In hydrating batch-wise mode, the unreacted nitrile is hydrated until the conversion reaches 99.9% or more, substantially 100%, the conversion showing the proportion of nitriles converted into an amide (A) among nitriles introduced into the previous continuous reactor (2). The time necessary for hydration in the batch-wise reactor (3) is usually about 0.2 hours to 2 hours.

By thus hydrating, an amide (A) can be obtained as reaction liquid containing substantially no nitrile. This reaction liquid contains unreacted water and inorganic acid, in addition to the intended amide (A), and HMBA generated by hydrolysis of the amide may be contained in slight amount.

An amide (A) is thus obtained as reaction liquid according to the process for the present invention, HMBA can be obtained by hydrolysis of the resultant amide (A). The amid (A) is usually hydrolyzed as reaction liquid intact.

For hydrolysis of the amide (A), for example, it may be advantageous that water (F) is added to the resultant reaction liquid and the mixture is heated. The amount of water (F) to be added is about 1-fold weight to 2-fold weight with respect to the inorganic acid (D) used in previous hydration. When sulfuric acid is used as the inorganic acid (D), ammonium sulfate and ammonium bisulfate may be added together with the water (F) [patent document 1: JP-A No. 2001-187779, paragraph nos. 0038, 0046 and 0052].

The hydrolysis temperature is usually 90° C. to 130° C. When lower than 90° C., there is tendency of insufficient hydrolysis. The boiling point of the reaction liquid is lower than 130° C. under atmospheric pressure in some cases, and in such cases, the liquid can be heated up to hydrolysis temperature over the boiling point under atmospheric pressure by heating under pressure, however, it is preferable to carry out hydrolysis at a temperature not higher than the boiling point of the reaction liquid under atmospheric pressure since an equipment for pressing is not necessary. Though lighter components of low boiling point may evaporate during the process of hydrolysis, it may be advantageous that the lighter components are purged during hydrolysis or after hydrolysis. The time necessary for the hydrolysis is usually about 2 hours to 5 hours.

The reaction liquid (A) may be extracted from the extraction tube (37) of the batch-wise reactor (3) used in previous hydration and transferred to another batch-wise reactor to perform hydrolysis, however, it may also be permissible that the reaction liquid is not extracted from the batch-wise reactor (3) used in previous hydration, and water (F) is added from a water introduction tube (38) without extraction, and the liquid is heated to cause hydrolysis. Since heat generated by hydrolysis of an amide is relatively small, heat removal can be carried out sufficiently by a low-capacity cooling apparatus even in the case of a batch-wise reactor.

By thus performing hydrolysis, 2-hydroxy-4-methylthiobutanoic acid (HMBA)(G) can be obtained as hydrolyzed liquid. The resultant HMBA is purified and used as feedstuff additives and the like.

### EXAMPLES

The present invention will be illustrated further in detail below by examples, but the present invention is not limited to these examples.

### Example 1

A continuous stirred tank reactor (2a) using one tank reactor body (20a) as the continuous reactor (2) as shown in FIG. 1 is used, and into this reactor (2a) is introduced, at 55° C., 2-hydroxy-4-methylthiobutanenitrile (B) at 131.20 g/hr (1 mol/hr) through a nitrile introduction tube (21) and 63% sulfuric acid aqueous solution (D′) at 116.76 g/hr (in terms of sulfuric acid; 0.75 mol/hr, water; 43.20 g/hr) through an inorganic acid aqueous solution introduction tube (23), respectively continuously, and under this condition, the nitrile is hydrated with an average residence time of 45 minutes (0.75 hours) while maintaining the internal temperature at 55° C. by removing heat by the jacket (23), and the hydrated reaction liquid (E) is continuously extracted by the extraction tube (27).

When 131.20 g (1 mol) of the nitrile is mixed with 116.76 g of 63% sulfuric acid, a mixing heat of 0.92 J is generated. When the nitrile is hydrated to give 2-hydroxy-4-methylthiobutaneamide, a hydration heat of 93.56 J per mol of the nitrile is generated, and when reacted at 55° C., about 90% of the nitrile is hydrated after 45 minutes, that is, the heat generation value due to the hydration reaction by this tank reactor body (20a) is 84.20 J per hour (=93.56 J×0.90). Thus, heat removal may be advantageously performed at a rate of 85.12 J/hr (=0.92+84.20) from this tank reactor (20a).

The hydrated reaction liquid (E) extracted is transferred to the hydrated reaction liquid storing tank (4) and stored at 55° C. In this storing tank (4), the hydrated reaction liquid (E) of an amount corresponding to 6 hours (1488 g) is stored. Also during storage for 6 hours, hydration progresses, leading to a conversion of 99%, and in this procedure, the hydration reaction progresses approximately at uniform rate, and the heat generation value generated during this is 50.52 J (=93.56 J×0.09×6), thus, heat removal may be advantageously performed at a rate of 8.42 J/hr (=50.52/6) from this storing tank (4).

The hydrated reaction liquid (E) corresponding to 6 hours stored on the hydrated reaction liquid storing tank (4) is transferred to an empty tank reactor body (30) constituting the batch-wise reactor (3) and filled therein while maintaining at 55° C., and after completion of filling, the temperature is maintained at 55° C. further for 1 hour, thereby, the unreacted nitrile contained in the hydrated reaction liquid (E) can be hydrated substantially at a conversion of 100%. In this procedure, the hydration reaction progresses approximately at uniform rate, and the heat generation value generated during this is 5.61 J (=93.56 J×0.01×6), thus, heat removal may be advantageously performed at a rate of 0.935 J/hr (=5.61 J/6 hrs) from this tank reactor (30).

### Example 2

A serial continuous stirred tank reactor (2b) using two continuous stirred tank reactors (2a) serially connected as the continuous reactor (2) as shown in FIG. 2 is used, and into this reactor (2b) is introduced a nitrile (B) at 131.20 g/hr (1 mol/hr) through a nitrile introduction tube (21) and 63% sulfuric acid aqueous solution (D′) at 116.76 g/hr (in terms of sulfuric acid; 0.75 mol/hr, water; 43.20 g/hr) through an inorganic acid aqueous solution introduction tube (23), respectively continuously, and under this condition, the hydrated reaction liquid (E) is continuously extracted through the extraction tube (27) so that the total average residence time of the two continuous stirred tank reactors (2a) is 45 minutes (0.75 hours), while maintaining the internal temperature at 55° C. by removing heat from the jacket (33) by the cooling apparatus (25), thereby hydrating the nitrile. Heat removal may be advantageously performed at a total heat removal rate of 85.12 J/hr from these two continuous stirred tank reactors (2a, 2a).

The hydrated reaction liquid (E) extracted through the extraction tube (27) of the serial continuous tank reactor (2b) is transferred directly to an empty tank reactor body (30) constituting the batch-wise reactor (3) and filled therein while maintaining at 55° C. over a period of 6 hours, and after completion of filling, the temperature is maintained at 55° C. further for 6 hours, thereby, the unreacted nitrile contained in the hydrated reaction liquid (E) is hydrated substantially at a conversion of 100%. In this procedure, the hydration reaction progresses approximately at uniform rate, and the heat generation value generated during this is 56.14 J (=93.56 J×0.1×6), thus, heat removal may be advantageously performed at a heat removal rate of 9.36 J/hr (=56.14 J/6 hrs) from this tank reactor (30).

### Example 3

A nitrile is hydrated by the same operation as in Example 1 excepting that a production equipment (1) using a tubular reactor (2c) as the continuous reactor (2) as shown in FIG. 3 is used instead of the production equipment shown in FIG. 1. In this production equipment (1), heat removal may be advantageously performed at a heat removal rate of 85.12 J/hr from the tubular reactor (2c), a heat removal rate of 8.42 J/hr from the storing tank (4) and a heat removal rate of 0.936 J/hr from the tank reactor (30), respectively.

### Example 4

A nitrile is hydrated by the same operation as in Example 2 excepting that a production equipment (1) using a tubular reactor (2c) as the continuous reactor (2) as shown in FIG. 4 is used instead of the production equipment shown in FIG. 2. In this production equipment, heat removal may be advantageously performed at a heat removal rate of 85.12 J/hr from the tubular reactor (2c), and heat removal may be advantageously performed at a total heat removal rate of 9.36 J/hr from the tank reactor (30).

### Example 5

A nitrile is hydrated by the same operation as in Example 1 excepting that a production equipment (1) using a loop reactor (2d) as the continuous reactor (2) as shown in FIG. 5 is used instead of the production equipment shown in FIG. 1. In this production equipment (1), heat removal may be advantageously performed at a heat removal rate of 85.12 J/hr from the loop reactor (2d), a heat removal rate of 8.42 J/hr from the storing tank (4) and a heat removal rate of 0.935 J/hr from the tank reactor (30), respectively.

### Example 6

A nitrile is hydrated by the same operation as in Example 2 excepting that a production equipment (1) using a loop reactor (2d) as the continuous reactor (2) as shown in FIG. 6 is used instead of the production equipment shown in FIG. 2. In this production equipment, heat removal may be advantageously performed at a heat removal rate of 85.12 J/hr from the loop reactor (2d), and heat removal may be advantageously performed at a heat removal rate of 9.36 J/hr from the tank reactor (30).

### Comparative Example 1

When 131.20 g of 2-hydroxy-4-methylthiobutanenitrile and 116.76 g of 63% sulfuric acid aqueous solution are introduced simultaneously into an empty tank reactor (30) each independently, the reaction progresses quickly, and after 1 minute, an amide is produced by hydration at a conversion of 90%. During this procedure, the total heat generation value is 85.12 J composed of a mixing heat of 0.92 J due to mixing and a heat generation value of 84.20 J due to hydration, and for maintaining the temperature at 55° C. during this operation, heat removal should be performed at a heat removal rate of 5107 J/hr since this total heat generation value of 85.12 J has to be removed in 1 minute.

1. A process for producing 2-hydroxy-4-methylthiobutaneamide by hydration of 2-hydroxy-4-methylthiobutanenitrile in the presence of an inorganic acid, comprising hydrating in continuous mode 2-hydroxy-4-methylthiobutanenitrile in the presence of an inorganic acid so as to give a conversion of 80% or more and 98% or less to obtain hydrated reaction liquid and hydrating in batch-wise mode unreacted 2-hydroxy-4-methylthiobutanenitrile contained in the resultant hydrated reaction liquid so as to give a conversion of 99.9% or more.

2. The process according to claim 1, wherein the inorganic acid is sulfuric acid and the use amount thereof is 0.5-fold mol to 1-fold mol with respect to 2-hydroxy-4-methylthiobutanenitrile.

3. The process according to claim 1, wherein the nitrile is hydrated in continuous mode using a continuous tank reactor, serial continuous tank reactor, tubular reactor or loop reactor.

4. The process according to claim 1 or 2, wherein the continuous mode hydration is carried out at 40° C. to 70° C. and the batch-wise mode hydration is carried out at 40° C. to 70° C.

5. A process for producing 2-hydroxy-4-methylthiobutanoic acid, comprising obtaining 2-hydroxy-4-methylthiobutaneamide by the process according to claim 1 and hydrolyzing the resultant 2-hydroxy-4-methylthiobutaneamide.
